(19) United States
(12) Patent Application Publication
(10) Pub. No.: US 2008/0214795 A1
(43) Pub. Date: Sep. 4, 2008

(54) Method of isolating antibodies by precipitation

(75) Inventors: Sundar Ramanan, Simi Valley, CA (US); Rosalind Stenson, Thousand Oaks, CA (US)

Correspondence Address: AMGEN INC., MAIL STOP 28-2-C, ONE AMGEN CENTER DRIVE, THOUSAND OAKS, CA, 91320-1799, US

(73) Assignee: Amgen Inc., Thousand Oaks, CA (US)

(21) Appl. No.: 12/069,996

(22) Filed: Feb. 13, 2008

Related U.S. Application Data
(60) Provisional application No. 60/901,851, filed on Feb. 14, 2007.

(51) Int. Cl. C07K 1/00

(52) U.S. Cl. 530/421

(57) ABSTRACT

Methods of isolating antibodies by precipitation are disclosed. Various precipitants that can be employed in the invention are also disclosed, with PEG being a preferred precipitant. In a representative embodiment of the invention, the pH of a solution comprising an antibody of interest is adjusted to ±0.5 pH unit of the pI of the antibody, a precipitant such as PEG is added and the antibody of interest is subsequently isolated from the resulting precipitate. The antibody can be further purified if desired or it can be resuspended in a buffer. The invention can be employed as an alternative to or in addition to chromatographic isolation methods, such as methods that employ affinity chromatography.

### FIELD OF THE INVENTION

The present invention relates to methods of isolating antibodies. More particularly, the invention relates to methods for isolating antibodies by precipitation. The isolation can be accomplished using PEG of various molecular weights as a precipitant.

### BACKGROUND OF THE INVENTION

Recent advances in cell culture technology for monoclonal antibody (MAb) production allow for titers of about 5 g/L or more, but these high titers present challenges to large-scale downstream processes. For example, when isolating large amounts of antibodies, one consideration is whether a preferred isolation method can be adapted to accommodate large scale operations. Another consideration is whether a preferred isolation method can be performed quickly and efficiently.

These considerations have been addressed by researchers in the field of antibody isolation. For example, some antibody isolation protocols employ an affinity-based purification step, such as a Protein A-based separation. While the use of an affinity-based purification approach facilitates the isolation of antibodies, it can also consume time and resources.

Although Protein A provides high selectivity, a Protein A column with a typical binding capacity of 25 g/L-resin for MAbs requires about 6 cycles for a standard 25 cm (L) by 1.6 m (ID) column to process a single 15,000 L bioreactor output volume. The cost of Protein A resin for this process can be quite high. The relatively large number of cycles required for acceptable purity levels can also increase the production cost. Moreover, higher titers are often accompanied by high cell culture density, which can result in culture fluid containing high concentrations of column-fouling nucleic acids and lipids. Without the regular use of appropriate column cleaning agents and operations, the lifetime of a Protein A column may be shortened, further increasing production costs.

Accordingly, a method of isolating antibodies to high levels of purity that saves time and cost, while at the same time being scalable and efficient, would be desirable.

### SUMMARY OF THE INVENTION

In one aspect, the present invention provides a method of isolating a monoclonal antibody from cell-free cell culture media. In one embodiment, the method comprises the steps of: (a) adjusting the pH of a volume of cell-free cell culture media comprising the antibody to within ±0.5 pH unit of the pI of the antibody; (b) incubating the volume of cell culture media with an aqueous PEG solution to form a mixture comprising an antibody precipitate and liquid culture media; (c) separating the antibody precipitate from the liquid culture media; and (d) resuspending the antibody precipitate in a resuspension buffer.

Continuing, the monoclonal antibody can be, for example, an IgG antibody. The adjusting step can be performed at a temperature between 2° C. and 8° C. The PEG can have a molecular weight of between 1.5 kD and 20 kD, for example 6 kD. The concentration of PEG in the aqueous PEG solution can be between 0.5% (w/v) and 30% (w/v), for example 10% (w/v). The incubating can be performed at a temperature selected from the group consisting of (a) between 2° C. and 8° C. and (b) room temperature. The incubating step can further comprise incubating the volume of cell culture media with a stabilizing compound, such as a stabilizing compound selected from the group consisting of glycine, arginine and sugars. Further, the incubation can be for a period of between 15 minutes and 24 hours, for example between 15 minutes and 2 hours or, for example, 30 minutes. The separating step can comprise, for example, (a) centrifuging the mixture to form the antibody precipitate and the liquid culture media; and (b) removing the liquid culture media from the antibody precipitate. In another embodiment, the separating can comprise filtering the mixture to form the antibody precipitate and the liquid culture media. Additionally, the resuspension buffer can have a pH of between 4.0 and 9.0. Further, the method can provide at least 70% recovery of antibodies.

In another aspect, the present invention provides a method of removing a protein contaminant from cell-free cell culture media. In one embodiment the method comprises the steps of (a) adjusting the pH of a volume of cell-free cell culture media comprising the protein contaminant to within ±0.5 pH unit of the pI of the protein contaminant; (b) incubating the volume of cell culture media with an aqueous PEG solution to form a mixture comprising a protein contaminant precipitate and liquid culture media; and (c) separating the protein contaminant precipitate from the liquid culture media.

Continuing, the monoclonal antibody can be, for example, an IgG antibody. The adjusting step can be performed at a temperature between 2° C. and 8° C. The PEG can have a molecular weight of between 1.5 kD and 20 kD, for example 6 kD. The concentration of PEG in the aqueous PEG solution can be between 0.5% (w/v) and 30% (w/v), for example 10% (w/v). The incubating can be performed at a temperature selected from the group consisting of (a) between 2° C. and 8° C. and (b) room temperature. Further, the incubation can be for a period of between 15 minutes and 24 hours, for example between 15 minutes and 2 hours or, for example, 30 minutes. The separating step can comprise, for example, (a) centrifuging the mixture to form the antibody precipitate and the liquid culture media; and (b) removing the liquid culture media from the antibody precipitate. In another embodiment, the separating can comprise filtering the mixture to form the antibody precipitate and the liquid culture media.

### DESCRIPTION OF THE DRAWINGS

FIG. 1 is a size exclusion chromatography (SEC) spectrum showing the results of a size exclusion chromatography analysis of an antibody isolated using a PEG precipitation method of the present invention (dots), and demonstrates that the antibody purified using PEG precipitation was of comparable purity to the same antibody purified using three chromatography steps (solid line).

FIG. 2 is a photograph of a gel depicting the results of a purity obtained using PEG precipitation step by SDS-PAGE. Lanes 1 through 4 corresponds to conditions when run under non-reducing conditions and Lanes 5 through 8 were run under reducing conditions. Lanes 1 and 5 correspond to molecular weight standards; Lanes 2 and 6 correspond to a PEG purified antibody sample; Lanes 3 and 7 correspond to PEG purified precipitate, further purified using one column; and Lanes 4 and 8 correspond to samples purified using a three column process.

FIG. 3A is a spectrum showing the secondary structure of Antibody 1, obtained using FTIR spectroscopy, after isolation of Antibody 1 using PEG precipitation followed by two chromatography steps (dots). An FTIR spectrum of Antibody 1 purified by three chromatography steps is shown as a solid line.

FIG. 3B is a spectrum showing the secondary structure of Antibody 1, obtained using Far UVCD spectroscopy, after isolation of Antibody I using PEG precipitation followed by two chromatography steps. A far UVCD spectrum of Antibody 1 purified by three chromatography steps is shown in solid line.

FIG. 4A is a spectrum showing the tertiary structure of Antibody 1 obtained using fluorescence spectroscopy, after isolation of Antibody 1 using PEG precipitation followed by two chromatography steps (dots). A fluorescence spectroscopy spectrum of Antibody 1 purified by a three column process is shown as a solid line.

FIG. 4B is a spectrum showing the tertiary structure of Antibody 1 obtained using UVCD spectroscopy, after isolation of Antibody 1 using PEG precipitation followed by two chromatography steps (dots). A CD spectroscopy spectrum of Antibody 1 purified using a three column process is shown as a solid line.

FIG. 5 is a plot showing the surface hydrophobicity of Antibody 1, after isolation of Antibody 1 using PEG precipitation followed by two chromatography steps (triangle). Surface hyrdophobicity of Antibody 1 purified using three chromatography steps is shown in plus symbol.

FIG. 6 is a plot showing the thermal stability of Antibody 1, after isolation of Antibody 1 using PEG precipitation followed by two chromatography steps (dots). Thermal stability of Antibody 1 purified using three chromatography steps in shown as a solid line.

### DETAILED DESCRIPTION OF THE INVENTION

### Definitions

For convenience, certain terms employed in the specification, examples, and claims are collected here.

As used in the present disclosure, including the claims, the terms “a,” “an” and “the” mean “one or more” unless the context clearly dictates otherwise. Thus, for example, reference to “an antibody” or “the antibody,” or “a protein contaminant” or “the protein contaminant,” is a reference to one or more antibodies and equivalents thereof, or to one or more protein contaminants.

As used herein, the term “antibody” means any recombinant or naturally-occurring intact antibody, e.g. an antibody comprising an antigen-binding variable region as well as a light chain constant domain (CL) and heavy chain constant domains. Also encompassed by the term are antibody fragments, or molecules including antibody fragments, including, but not limited to, Fab, Fab′, F(ab′)₂, Fv and Fc fragments. The term “antibody” specifically encompasses fusion proteins such as Fc fusion proteins, peptibodies and other chimeric antibodies. Consistent with the use of the terms “a” and “an” in the present disclosure, the term “an antibody” specifically includes a population of an antibody of interest and is not limited to a single antibody. The term “antibody” specifically encompasses both monoclonal and polyclonal antibodies.

As used herein, the term “cell-free cell culture media” means cell culture media from which whole cells have been removed, for example by filtration. Cell-free cell culture media can be, but need not, be clarified.

### Method of Isolating an Antibody

In one aspect, the present invention comprises a method of isolating an antibody from cell-free cell culture media. In various embodiments the antibody can be an IgG antibody, for example an IgG1, IgG2, IgG3 or IgG4 antibody. Also encompassed by the term antibody are antibody fragments, chimeric antibodies, fusion proteins such as Fc fusion proteins and peptibodies.

The method can be applied in any antibody production regimen, for example in isolating antibodies secreted from cells into culture media. In the context of the present invention, the cell culture media is preferably cell-free and consequently is preferably free of any unlysed, whole cells. Cells can be removed from the cell culture media by employing any conveniently available method, for example by filtration or by centrifugation. The cell-free culture media can, but need not, be clarified. When the media is clarified, it can be clarified by employing any conveniently available method, for example depth filtration, microfiltration or a combination of techniques. As noted above, although the present invention is described in terms of isolating an antibody of interest, it will be understood that the method encompasses isolating a population of a particular antibody of interest.

Continuing, the pH of a volume of cell-free cell culture media comprising the antibody is adjusted to within ±0.5 pH unit of the pI of the antibody. The pI of the antibody can be readily determined using one of the various methods of determining pI known to those of ordinary skill in the art. In a preferred embodiment, the pI is determined by performing capillary isoelectric focusing (cIEF) on a sample comprising the antibody and measuring the pI. Methods for performing cIEF are known (see, e.g., Kundu & Fenters, (1995) J. Capillary Electrophor. 2(6):273-77) and materials for performing cIEF are commercially available. As noted herein, the method can be applied to any volume of cell-free cell media. The pI of the antibody can be determined before the adjusting step described below or the determination of the pI can be carried out as a step in the disclosed method.

Once the pI has been determined, the pH of the cell-free cell culture media is adjusted to within ±0.5 pH unit of the pI of the antibody of interest. The adjusting can be carried out in any convenient fashion, for example by adding aliquots of an acidic or basic solution to the media until the pH of the media falls within the acceptable pH range. It is preferable to achieve and maintain a media pH equal to the pI of the antibody of interest, however precisely matching the pH and pI values is not required and the invention encompasses pH values within ±0.5 pH unit of the pI of the antibody of interest. In fact, in some cases it may be desirable to set the pH to a value that is close to, but not exactly, the pI of the antibody.

After adjusting the pH of the cell culture media to within ±0.5 pH unit of the pI of the antibody, the volume of cell culture media is incubated with an aqueous polyethylene glycol (PEG) solution to form a mixture comprising an antibody precipitate and liquid culture media. The aqueous PEG solution comprises at least water and PEG, but can comprise other components as well. For example, it may be desirable to buffer the aqueous solution to a pH close to or matching the pI of the antibody. Preferably, the PEG of the aqueous solution has a molecular weight of between 0.25 kD and 50 kD, for example 4 kD, 6 kD or 8 kD, but can be of any molecular weight. PEG polymers having molecular weights of between 0.25 kD and 50 kD are commercially available, for example from Alfa Aesar of Ward Hill, Mass., and can be employed in the practice of the present invention. The concentration of PEG in the aqueous solution is preferably between 0.5% (w/v) and 30% (w/v), for example 5% (w/v), 10% (w/v) or 15% (w/v). Any form of PEG can be employed in this and the other methods of the present invention, for example linear PEGs and branched or multi-armed PEGs, as well as derivatized PEGs.

The weight of the PEG employed in all embodiments of the present invention can vary with the nature of the antibody to be isolated. For example, isolation of a first antibody may be best achieved using PEG having a first molecular weight, while isolation of a second antibody may be best achieved using PEG having a second molecular weight. Those of ordinary skill in the art will recognize that an optimal PEG weight for a given antibody and set of precipitation conditions can be readily ascertained by varying the PEG weight, performing the method of the present disclosure, measuring the amounts of the antibody precipitated for each PEG weight, and selecting the PEG weight that provides the desired level of isolated antibody.

Similarly, the concentration of PEG in the aqueous PEG solution can also be varied with the nature of the antibody to be isolated. For example, isolation of a first antibody may be best achieved using an aqueous solution having a first concentration of PEG, while isolation of a second antibody may be best achieved using an aqueous solution having a second concentration of PEG. Those of ordinary skill in the art will recognize that an optimal concentration of PEG in the aqueous solution for a given antibody and set of precipitation conditions can be readily ascertained by varying the PEG concentration in the solution, performing the method of the present disclosure, measuring the amounts of the antibody precipitated for each PEG concentration, and selecting the PEG concentration that provides the desired level of isolated antibody.

The adjusting can be carried out at any temperature between 2° C. and 30° C., such as a temperature of between 15° C. and 25° C. or a temperature between 2° C. and about 8° C.

The volume of cell culture and the aqueous PEG solution can incubated for any period of time, but the incubation preferably is for a period of time between 15 minutes and 24 hours, for example between 15 minutes and 2 hours or, for example, 30 minutes. Again, the length of the incubation can vary with the antibody to be isolated and can be optimized by varying the incubation time for a given set of conditions (e.g., PEG concentration, PEG weight, etc.), measuring the amounts of the antibody that is precipitated for each incubation period and selecting the incubation period that provides the optimal or desired level of isolated antibody.

Over the course of the incubation period, the mixture can be mixed continuously, at regular intervals, only a desired number of times or not at all. Mixing is not required, but those of skill in the art will recognize when, in the practice of the present invention, mixing may be desirable in the formation of the antibody precipitate and the liquid culture media.

The incubation can be carried out at any temperature found to be conducive to the formation of the antibody precipitate and the liquid culture media. For example, the incubation can be performed at a temperature between 2° C. and 8° C. or at room temperature. Indeed, one advantage of the present invention is the ability to perform the incubation step at room temperature, with no need to keep the mixture refrigerated or even set to a particular temperature.

The incubation of the cell culture media with the aqueous PEG solution forms a mixture comprising an antibody precipitate phase and a liquid culture media phase. Following the incubation, the mixture can then be separated into the antibody precipitate and the liquid culture media by employing any convenient approach. In one embodiment, the mixture is centrifuged. In this embodiment, the antibody precipitate collects at the bottom of the vessel in which the mixture is centrifuged, while the liquid culture media, which comprises less antibody precipitate than does the collected antibody precipitate, remains supernatant. Following the centrifugation, the liquid culture media can be removed from the antibody precipitate, for example by decanting or by aspiration.

In another embodiment, the mixture can be separated into its antibody precipitate and the liquid culture media phases by filtration. In one example, the mixture is passed through a filter under suction and the antibody precipitate is collected on the filter, leaving the liquid culture media to pass through the filter into a collection vessel. In another example, the mixture is passed through a filter under the force of gravity. In yet another embodiment, the mixture is passed through a filter under pressure, using a plunger-like device to force the mixture through the filter.

Following the separation of the antibody precipitate from the liquid culture media, the antibody precipitate can optionally be washed with a buffer. A goal of the optional washing step may be to remove residual liquid culture medium from the antibody precipitate. The optional washing can comprise simply contacting a wash buffer with the antibody precipitate and then removing the wash buffer by aspirating or decanting the buffer away from the antibody precipitate. As noted, washing the antibody precipitate is optional, but one case in which it may be desirable to wash the antibody precipitate is when no further isolation or polishing steps will be performed subsequent to the methods of the present invention.

Following the separation of the antibody precipitate from the liquid culture media, the antibody precipitate can be resuspended in a buffer. The resuspension buffer can be any suitable buffer and will depend, at least in part, on the properties of the antibody being isolated. Preferably, the resuspension buffer has a pH of between 4.0 and 9.0. One example of a resuspension buffer is an acetate buffer at pH 5.0.

Following resuspension of the antibody precipitate in the resuspension buffer, the resuspension buffer comprising the antibody can optionally be further processed by filtration to preserve the sterility of the solution and prepare it for storage. For example, the resuspension buffer comprising the antibody can be filtered through a 0.22 micron filter to remove any potential bacterial contamination.

The filtered or non-filtered resuspension buffer comprising the antibody can be stored at 4° C. for later use and/or further polishing, as desired.

### Method of Removing a Protein Contaminant

In another aspect of the present invention, a method of removing a protein contaminant from cell-free cell culture media is disclosed. The method can be applied in any scenario in which a protein contaminant is known or suspected to be present in a volume of cell culture media. The method can be employed, for example, in a quality control protocol to assess the purity of a product, or as a step in the isolation of a particular component of a volume of cell culture media.

As stated previously, in the context of the present invention, the cell culture media is preferably cell-free and consequently free of any unlysed, whole cells. Cells can be removed from the cell culture media by employing any conveniently available method, for example by filtration or by centrifugation. Further, the cell-free culture media can, but need not, be clarified. If desired, the media can be clarified by employing any conveniently available method, for example depth filtration and microfiltration.

Continuing, the pH of a volume of cell-free cell culture media comprising the antibody is adjusted to within ±0.5 pH unit of the pI of the protein contaminant to be removed from the volume of cell-free culture media. The pI of the protein contaminant can be readily determined using one of the various methods of determining pI known to those of ordinary skill in the art. In a preferred embodiment, the pI is determined by performing capillary isoelectric focusing (cIEF) on a sample comprising the protein contaminant and measuring the pI. The pI of the protein contaminant can be determined before the adjusting step or the determination of the pI can be carried out as a step in the disclosed method.

Once the pI has been determined, the pH of the cell-free cell culture media is adjusted to within ±0.5 pH unit of the pI of the protein contaminant. The adjusting can be carried out in any convenient fashion, for example by adding aliquots of an acidic or basic solution to the media until the pH of the media falls within the acceptable pH range. It is preferable to achieve and maintain a media pH equal to the pI of the protein contaminant, however precisely matching the pH and pI values is not required and the invention encompasses pH values within ±0.5 pH unit of the pI of the protein contaminant. In fact, in some cases it may be desirable to set the pH to a value that is close to, but not exactly, the pI of the protein contaminant, under which conditions it may be possible to precipitate several protein contaminants having similar properties.

After adjusting the pH of the cell culture media to within ±0.5 pH unit of the pI of the protein contaminant, the volume of cell culture media is incubated with an aqueous polyethylene glycol (PEG) solution to form a mixture comprising a protein contaminant precipitate and liquid culture media. The aqueous PEG solution comprises at least water and PEG, but can comprise other components as well. As in the embodiment comprising the isolation of an antibody described herein, it may be desirable to buffer the aqueous solution to a pH close to or matching the pI of the protein contaminant. Again, the PEG of the aqueous solution has a molecular weight of between 0.25 kD and 50 kD, for example 4 kD, 6 kD or 8 kD, but can be of any molecular weight, are commercially available. The concentration of PEG in the aqueous solution is preferably between 0.5% (w/v) and 30% (w/v), for example 5% (w/v), 10% (w/v) or 15% (w/v). Further, the cell culture media and the aqueous PEG solution can incubated for any period of time, but the incubation preferably is for a period of time between 15 minutes and 24 hours, for example between 15 minutes and 2 hours or, for example, 30 minutes.

As in the embodiment of the present invention direct to isolating an antibody, the weight of the PEG employed in the present invention can vary with the nature of the protein contaminant to be isolated. For example, isolation of a first protein contaminant may be best achieved using PEG having a first molecular weight, while isolation of a second protein contaminant may be best achieved using PEG having a second molecular weight. The precise weight of PEG employed can be optimized by employing routine optimization procedures, such as those described herein above.

The concentration of PEG in the aqueous PEG solution can also be varied with the nature of the protein contaminant to be isolated. For example, isolation of a first protein contaminant may be best achieved using an aqueous solution having a first concentration of PEG, while isolation of a second protein contaminant may be best achieved using an aqueous solution having a second concentration of PEG. Again, the concentration of PEG in the aqueous PEG solution can be optimized through routine procedures, as described herein.

The adjusting can be carried out at any temperature between 2° C. and 30° C., such as a temperature of between 15° C. and 25° C. or a temperature between 2° C. and 8° C.

The volume of cell culture with the aqueous PEG solution can incubated for any period of time, but the incubation preferably is for a period of time between 15 minutes and 24 hours for example, 30 minutes. Again, the length of the incubation can vary with the protein contaminant to be removed and can be optimized using standard procedures.

Over the course of the incubation period, the mixture can be mixed continuously, at regular intervals, only a desired number of times or not at all. Mixing is not required, but in some cases may assist in the formation of the protein contaminant precipitate and the liquid culture media.

The incubation can be carried out at any temperature found to be conducive to the formation of the protein contaminant precipitate and the liquid culture media, for example at a temperature between 2° C. and 8° C., or at room temperature.

The incubation of the cell culture media with the aqueous PEG solution forms a mixture comprising a protein contaminant precipitate phase and a liquid culture media phase. Following the incubation, the mixture can then be separated into the protein contaminant precipitate and the liquid culture media by employing any convenient approach. In a preferred embodiment, the mixture is centrifuged to precipitate the protein contaminant and the liquid culture media, which will be free of the protein contaminant, is removed from the protein contaminant precipitate, for example by decanting or by aspiration. Alternatively, the mixture can be separated into its protein contaminant precipitate and the liquid culture media phases by filtration.

After the protein contaminant has been removed from the liquid culture media, the media can be stored cold for subsequent processing, for example by employing the present invention to isolate an antibody from the liquid culture media.

It is noted that all embodiments of the present invention can be employed on any scale. For example, the present invention can be applied to large scale antibody production operations in which antibodies or impurities are isolated from tens, hundreds or thousands of liters of cell culture media. In another example, the present invention can be employed on a smaller scale, for example in bench-top scale operations in which antibodies or impurities are isolated from volumes on the order of several liters of media or even volumes of less than a liter of media.

### EXAMPLES

The following Examples have been included to illustrate embodiments of the present invention. Various aspects of the following Examples are described in terms of techniques and procedures found or contemplated by the present inventors to work well in the practice of the present invention. The following Examples are intended to be exemplary only and numerous changes, modifications and alterations can be employed without departing from the spirit and scope of the invention.

### Example 1

### Isolation of MAbs by PEG Precipitation and Two Chromatography Steps

Cell culture media containing MAb was harvested using a combination of centrifugation, depth filtration and membrane filtration. The cell debris-free media, henceforth referred to herein as “clarified cell culture media,” was then stored at 2-8° C. until the initiation of the precipitation step.

A 37.5% (w/v) stock solution of polyethylene glycol 6000 (PEG 6000) (Alfa Aesar, Ward Hill, Mass., USA) was added to the clarified cell culture media to produce a PEG 6000 concentration of 10% (w/v) in the final suspension. The suspension was mixed completely for a minimum of 30 minutes. The temperature during the mixing was maintained at 2-8° C. After the completion of the precipitation, the IgG rich precipitate was separated from the supernatant using a centrifuge operating at 3000 g, or by filtration. The supernatant was discarded. The antibody-containing precipitate can be stored and used after the PEG precipitation step or, if desired, it can be processed further.

The antibody-containing precipitate was resuspended in 30 mM sodium acetate, pH 5.0, and was loaded onto a CM Sepharose™ Fast Flow (GE Healthcare, Piscataway, N.J.) column which had been previously equilibrated with 30 mM sodium acetate, pH 5.0. During the loading step residual PEG6000 flowed through the column. The level of unbound PEG6000 present in the column was reduced further using a wash step using the CM Sepharose™ equilibration buffer (30 mM sodium acetate, pH, 5.0). The bound IgG was then eluted from the column using a 200 mM sodium chloride, 30 mM sodium acetate, pH 5.0 and collected. The CM Sepharose™ eluate was filtered through a nanofilter to remove any viral particles that might be present. Following the viral filtration step, the conductivity of the collected pool was conditioned using 800 mM sodium chloride, 30 mM sodium acetate, pH 5.0. The conditioned load was further loaded on to a Phenyl Sepharose™ Fast Flow (hi sub) column to remove any residual impurities. The isolated antibody was concentrated using a 30 kD membrane to a final concentration of about 70 g/L.

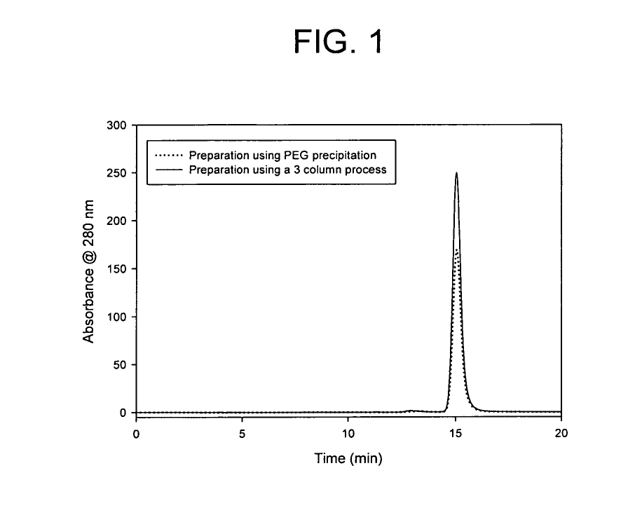

FIG. 1 depicts the results of a size exclusion chromatography analysis of the purified antibody using the above protocol and demonstrates that the antibody isolated using PEG precipitation was of comparable purity to the same antibody isolated using three chromatography steps.

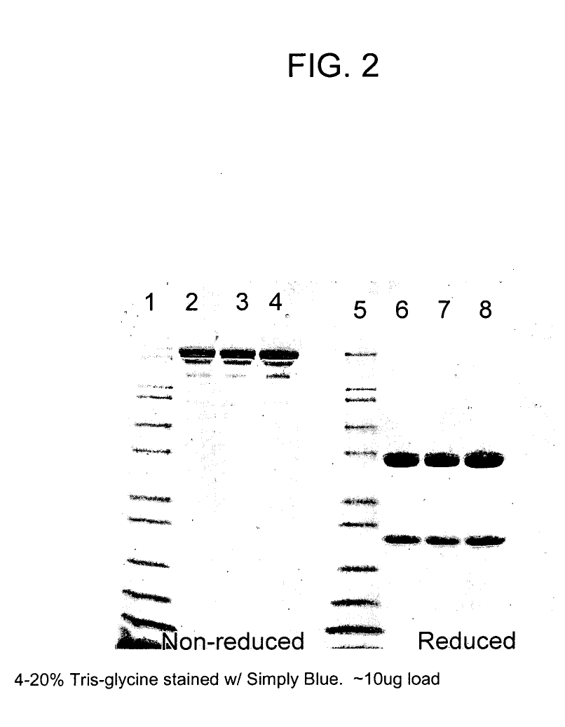

FIG. 2 depicts the results from SDS-PAGE of the isolated antibodies using the above protocol and demonstrates that the antibody isolated using PEG precipitation was of comparable purity to the same antibody isolated using three chromatography steps.

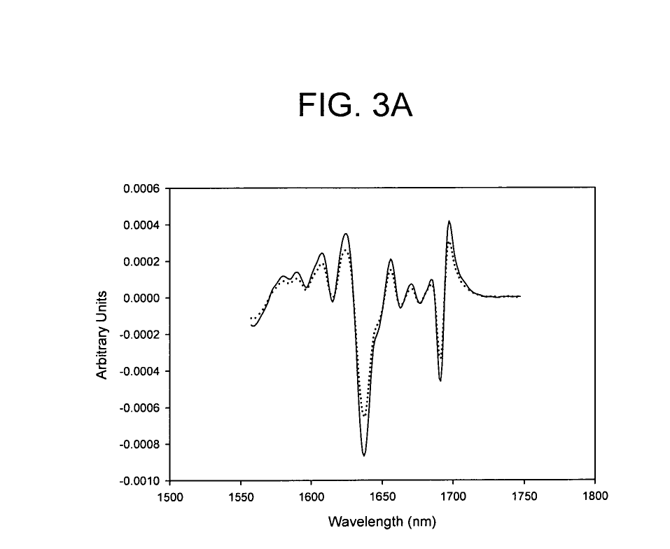

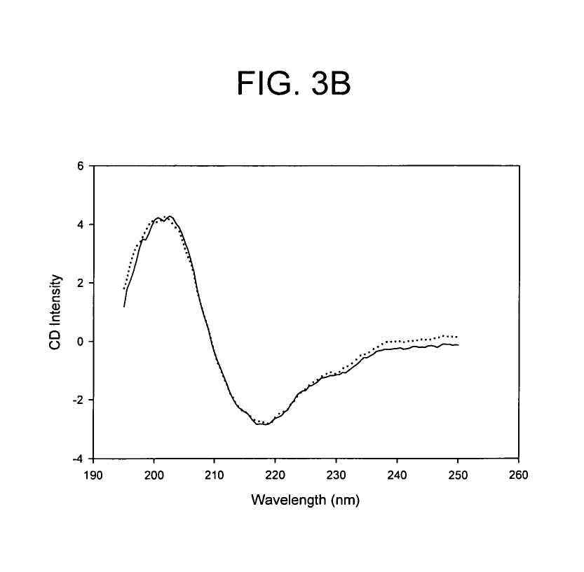

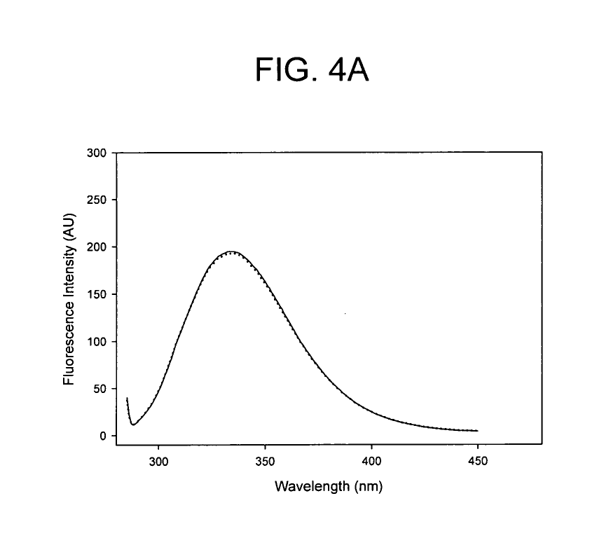

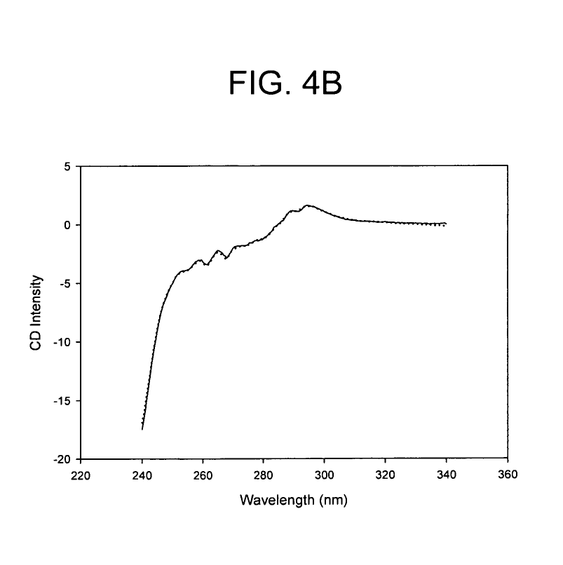

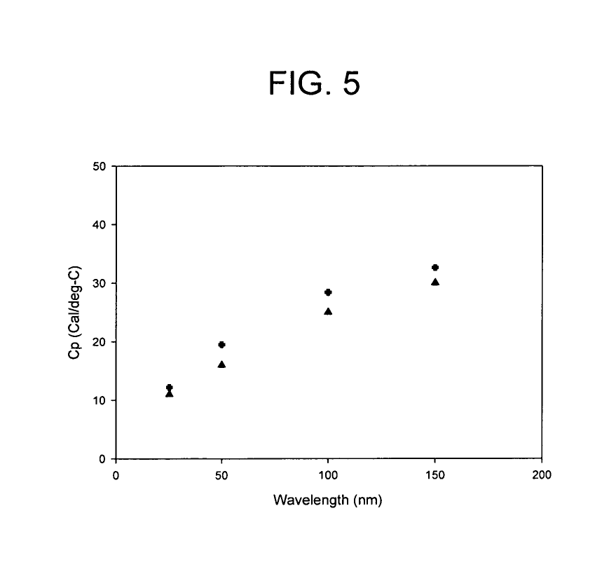

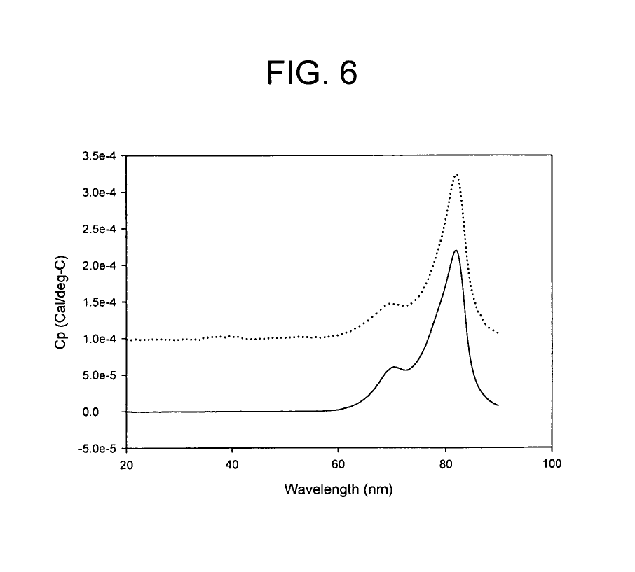

FIGS. 3 through 6 confirmed the results presented in FIGS. 1 and 2 through further characterization of the isolated antibody using FT and Far UV spectroscopy (FIGS. 3A and 3B), fluorescence and near UVCD spectroscopy (FIGS. 4A and 4B), surface hydrophobicity analysis (FIG. 5) and thermal stability analysis (FIG. 6)

### Example 2

### Isolation of MAbs by PEG Precipitation, Two Chromatography Steps and a Viral Kill Step

Cell culture media containing MAb antibodies was harvested using a combination of centrifugation, depth filtration and membrane filtration. The cell debris-free media, henceforth referred to herein as “clarified cell culture media,” was then stored at 2-8° C. until the initiation of the precipitation step.

A 37.5% (w/v) stock solution of polyethylene glycol 6000 (PEG 6000) (Alfa Aesar, Ward Hill, Mass., USA) was added to the clarified cell culture media to produce a PEG 6000 concentration of 10% (w/v) in the final suspension. The suspension was mixed completely for a minimum of 30 minutes. The temperature during the mixing was maintained at 2-8° C. After the completion of the precipitation, the IgG rich precipitate was separated from the supernatant using a centrifuge operating at 3000 g, or by filtration. The supernatant was discarded. The antibody-containing precipitate can be stored and used after the PEG precipitation step or, if desired, it can be processed further.

The antibody-containing precipitate was resuspended in 30 mM sodium acetate, pH 5.0 and mixed until the precipitate was completely dissolved in solution. The pH of this solution was lowered to 3.6±0.1 using 10% acetic acid. After holding the solution at the lowered pH for about 1 hour, the pH was slowly raised to a final pH of 5.0 using 1M Tris-Base.

The solution was then filtered using a series of depth filtration and a microfilration steps. In addition, prior to further processing the filtered solution was diluted using DI-water to lower the conductivity prior to loading on to a CM Sepharose™ Fast Flow column which had been previously equilibrated with 30 mM sodium acetate, pH 5.0. During the loading step residual PEG6000 flowed through the column. The level of unbound PEG6000 present in the column was reduced further through a wash step using the CM Sepharose™ equilibration buffer (30 mM sodium acetate, pH, 5.0). The bound IgG was then eluted from the column using a 200 mM sodium chloride, 30 mM sodium acetate, pH 5.0. The CM Sepharose™ eluate was filtered using a nanofilter to remove any viral particles present. The low-pH hold step can be performed at either before or after the 2^{nd} or 3^{rd} chromatography steps. After the viral filtration step the conductivity of the product pool was conditioned using 800 mM sodium chloride, 30 mM sodium acetate, pH 5.0. The conditioned load was then loaded on to a Phenyl Sepharose™ Fast Flow (hi sub) column to remove any residual impurities. The isolated product was concentrated using 30 kD membrane to a final concentration of about 70 g/L.

### Example 3

### Isolation of MAbs by PEG Precipitation, a Chromatography Step and a Membrane Chromatography Step

Cell culture media containing MAb is harvested using a combination of centrifugation, depth filtration and membrane filtration. The cell debris-free media, henceforth referred to herein as “clarified cell culture media,” is then stored at 2-8° C. until the initiation of the precipitation step.

A 37.5% (w/v) stock solution of polyethylene glycol 6000 (PEG 6000) (Alfa Aesar, Ward Hill, Mass., USA) is added to the clarified cell culture media to produce a PEG 6000 concentration of 10% (w/v) in the final suspension. The suspension is mixed completely for a minimum of 30 minutes. The temperature during the mixing is maintained at 2-8° C. After the completion of the precipitation, the IgG rich precipitate is separated from the supernatant using a centrifuge operating at 3000 g, or by filtration. The supernatant is discarded. The antibody-containing precipitate can be stored and used after the PEG precipitation step or, if desired, it can be processed further.

The antibody-containing precipitate is resuspended in 30 mM sodium acetate, pH 5.0, and is loaded onto a CM Sepharose™ Fast Flow (GE Healthcare, Piscataway, N.J.) column which has been previously equilibrated with 30 mM sodium acetate, pH 5.0. During the loading step any residual PEG6000 flows through the column. The level of unbound PEG6000 present in the column is reduced further using a wash step using the CM Sepharose™ equilibration buffer (30 mM sodium acetate, pH, 5.0). The bound IgG is then eluted from the column using a 200 mM sodium chloride, 30 mM sodium acetate, pH 5.0 and collected. The CM Sepharose™ eluate is filtered through a nanofilter to remove any viral particles that might be present. After the viral filtration step the product pool is loaded onto a negatively-charged membrane filter to remove any residual impurities. The isolated antibody is then concentrated using 30 kD membrane.

### Example 4

### Isolation of MAbs by PEG Precipitation, a Chromatography Step, a Membrane Chromatography Step and a Viral Kill Step

Cell culture media containing MAb is harvested using a combination of centrifugation, depth filtration and membrane filtration. The cell debris-free media, henceforth referred to herein as “clarified cell culture media,” is then stored at 2-8° C. until the initiation of the precipitation step.

A 37.5% (w/v) stock solution of polyethylene glycol 6000 (PEG 6000) (Alfa Aesar, Ward Hill, Mass., USA) is added to the clarified cell culture media to produce a PEG 6000 concentration of 10% (w/v) in the final suspension. The suspension is mixed completely for a minimum of 30 minutes. The temperature during the mixing is maintained at 2-8° C. After the completion of the precipitation, the IgG rich precipitate is separated from the supernatant using a centrifuge operating at 3000 g, or by filtration. The supernatant is discarded. The antibody-containing precipitate can be stored and used after the PEG precipitation step or, if desired, it can be processed further.

The antibody-containing precipitate is resuspended in 30 mM sodium acetate, pH 5.0 and mixed until the precipitate is completely dissolved in solution. The pH of this solution is lowered to 3.6±0.1 using 10% acetic acid. After holding the solution at the lowered pH for about 1 hour, the pH is slowly raised to a final pH of 5.0 using 1M Tris-Base.

The solution is then filtered using a series of depth filtration and a microfilration steps. In addition, prior to further processing the filtered solution is diluted using DI-water to lower the conductivity prior to loading on to a CM Sepharose™ Fast Flow column which has been previously equilibrated with 30 mM sodium acetate, pH 5.0. During the loading step any residual PEG6000 flows through the column. The level of unbound PEG6000 present in the column is reduced further through a wash step using the CM Sepharose™ equilibration buffer (30 mM sodium acetate, pH, 5.0). The bound IgG is then eluted from the column using a 200 mM sodium chloride, 30 mM sodium acetate, pH 5.0. The CM Sepharose™ eluate is filtered using a nanofilter to remove any viral particles present. The low-pH hold step can be performed at either before or after the 2^{nd} or 3^{rd} chromatography or membrane chromatography steps. After the viral filtration step the product pool is loaded onto a negatively-charged membrane filter to remove any residual impurities. The isolated antibody is then concentrated using 30 kD membrane.

What is claimed is:

1. A method of isolating a monoclonal antibody from cell-free cell culture media, the method comprising the steps of:
(a) adjusting the pH of a volume of cell-free cell culture media comprising the antibody to within ±0.5 pH unit of the pI of the antibody;
(b) incubating the volume of cell culture media with an aqueous PEG solution to form a mixture comprising an antibody precipitate and liquid culture media;
(c) separating the antibody precipitate from the liquid culture media; and
(d) resuspending the antibody precipitate in a resuspension buffer.

2. The method of claim 1, wherein the monoclonal antibody is an IgG antibody.

3. The method of claim 1, wherein the adjusting is performed at a temperature between 2° C. and 8° C.

4. The method of claim 1, wherein the PEG has a molecular weight of between 1.5 kD and 20 kD.

5. The method of claim 4, wherein the PEG has a molecular weight of 6 kD.

6. The method of claim 1, wherein the concentration of PEG in the aqueous PEG solution is between 0.5% (w/v) and 30% (w/v).

7. The method of claim 6, wherein the concentration of PEG in the aqueous PEG solution is 10% PEG (w/v).

8. The method of claim 1, wherein the incubating is performed at a temperature selected from the group consisting of (a) between 2° C. and 8° C. and (b) room temperature.

9. The method of claim 1, wherein the incubating of step (b) further comprises incubating the volume of cell culture media with a stabilizing compound.

10. The method of claim 9, wherein the stabilizing compound is selected from the group consisting of glycine, arginine and sugars.

11. The method of claim 1, wherein the incubation is for a period of between 15 minutes and 24 hours.

12. The method of claim 11, wherein the incubation is for a period of between 15 minutes and 2 hours.

13. The method of claim 12, wherein the incubation is for a period of 30 minutes.

14. The method of claim 1, wherein the separating comprises:
(a) centrifuging the mixture to form the antibody precipitate and the liquid culture media; and
(b) removing the liquid culture media from the antibody precipitate.

15. The method of claim 1, wherein the separating comprises filtering the mixture to form the antibody precipitate and the liquid culture media.

16. The method of claim 1, wherein the resuspension buffer has a pH of between 4.0 and 9.0.

17. The method of claim 1, wherein the method provides at least 70% recovery of antibodies.

18. A method of removing a protein contaminant from cell-free cell culture media, the method comprising the steps of:
(a) adjusting the pH of a volume of cell-free cell culture media comprising the protein contaminant to within ±0.5 pH unit of the pI of the protein contaminant;
(b) incubating the volume of cell culture media with an aqueous PEG solution to form a mixture comprising a protein contaminant precipitate and liquid culture media; and
(c) separating the protein contaminant precipitate from the liquid culture media.

19. The method of claim 18, wherein the adjusting is performed at a temperature between 2° C. and 8° C.

20. The method of claim 18, wherein the PEG has a molecular weight of between 1.5 kD to 20 kD.

21. The method of claim 20, wherein the PEG has a molecular weight of 6 kD.

22. The method of claim 18, wherein the concentration of PEG in the aqueous PEG solution is between 0.5% (w/v) and 30% (w/v).

23. The method of claim 22, wherein the concentration of PEG in the aqueous PEG solution is 10% PEG (w/v).

24. The method of claim 18, wherein the incubating is performed at a temperature selected from the group consisting of (a) between 2° C. and 8° C. and (b) room temperature.

25. The method of claim 18, wherein the incubation is for a period of between 15 minutes and 24 hours.

26. The method of claim 25, wherein the incubation is for a period of between 15 minutes and 2 hours.

27. The method of claim 26, wherein the incubation is for a period of 30 minutes.

28. The method of claim 18, wherein the separating comprises:
(a) centrifuging the mixture to form the protein contaminant precipitate and the liquid culture media; and
(b) removing the liquid culture media from the protein contaminant precipitate.

29. The method of claim 18, wherein the separating comprises filtering the mixture to form the protein contaminant precipitate and the liquid culture media.
